(19) United States
(12) Patent Application Publication
(10) Pub. No.: US 2024/0399138 A1
(43) Pub. Date: Dec. 5, 2024

(54) A Wirelessly Powered, Battery-Less Closed Loop Biopotential Recording IC for Implantable Medical Applications

(71) Applicant: The Regents of the University of California, Oakland, CA (US)

(72) Inventors: Jaeeun Jang, Los Angeles, CA (US); Aydin Babakhani, Los Angeles, CA (US)

(73) Assignee: The Regents of the University of California, Oakland, CA (US)

(21) Appl. No.: 18/697,858

(22) PCT Filed: Oct. 4, 2022
(86) PCT No.: PCT/US2022/077550

§ 371 Date: Apr. 2, 2024

Related U.S. Application Data
(60) Provisional application No. 63/252,104, filed on Oct. 4, 2021.

(51) Int. Cl. A61N 1/02; A61N 1/372; A61N 1/378; G16H 40/67

(52) U.S. Cl. CPC A61N 1/025; A61N 1/37229; A61N 1/37235; A61N 1/378; G16H 40/67

(57) ABSTRACT

Systems and methods for biopotential recording integrated circuits are illustrated. One embodiment includes a wireless receiver configured to receive a first radio frequency (RF) signal; one or more wireless transmitters; and a processing circuitry, comprising: a power harvesting circuit configured to harvest energy from the first RF signal; a clock recovery circuit configured to extract a clock signal from the first RF signal; at least one sensing electrode configured to record an electric signal as at least one of a voltage, current, and electric charge; and an analog-to-digital converter (ADC) communicatively coupled to the clock recovery circuit.

### CROSS-REFERENCE TO RELATED APPLICATIONS

The current application claims the benefit of and priority under 35 U.S.C. § 119(e) to U.S. Provisional Patent Application No. 63/252,104, titled “Wirelessly Powered, Battery-less Closed Loop Biopotential Recording IC for Implantable Medical Applications,” filed Oct. 4, 2021, the disclosure of which is hereby incorporated by reference in its entirety for all purposes.

### FIELD OF THE INVENTION

The present invention relates to wirelessly powered integrated circuit (IC) utilizing on-chip antennas and separate low dropout voltage regulators for a more precise power delivery and accurate cardiac and neural monitoring while maintaining a size as small as possible.

### BACKGROUND

The heart is an important muscle organ in humans and many other animals that is responsible for circulating blood through the circulatory system. The human heart is divided into four chambers, two upper atria, and two lower ventricles, organized into a left and right pairing of an atrium and a ventricle. In a healthy heart, the chambers contract and relax in a synchronized fashion, referred to as a “beat,” in order to pump blood through the network of blood vessels.

Arrhythmia is the condition where the patient's heart beats at an irregular rate, and serious cases could lead to strokes or heart failures. Pacemakers are life-saving devices for patients suffering from arrhythmia. Up to 250,000 pacemakers are implanted in patients in the United States each year. Pacemakers may stimulate heart muscles such that regular heart rhythm can be restored. Leadless pacemakers are a type of pacemaker that is small and self-contained, and can be inserted in the right ventricle of the heart.

Wireless power transmission refers to the transmission of electrical energy without wires as a physical connection. There are two main categories of wireless power techniques: near field and far field. An example of near field wireless power technique is inductive coupling, where power is transferred over a short distance through coils of wire or electric fields using capacitive coupling between metal electrodes.

### SUMMARY OF THE INVENTION

Systems and methods for biopotential recording integrated circuits are illustrated. One embodiment includes a sensing device. The sensor device includes a wireless receiver configured to receive a first radio frequency (RF) signal. The sensor device includes one or more wireless transmitters, wherein at least one wireless transmitter is configured to transmit a second RF signal, and wherein the first RF signal and the second RF signal have different frequencies. The sensor device includes a processing circuitry. The processing circuitry includes a power harvesting circuit configured to harvest energy from the first RF signal. The processing circuitry includes a clock recovery circuit configured to extract a clock signal from the first RF signal. The processing circuitry includes at least one sensing electrode configured to record an electric signal as at least one of a voltage, current, and electric charge. The processing circuitry includes an analog-to-digital converter (ADC) communicatively coupled to the clock recovery circuit, wherein the ADC is configured to convert the electric signal into a digital signal, wherein the clock signal is used to synchronize at least one wireless transmitter with the conversion of the electrical signal into the digital signal, and wherein output from the ADC is serialized and transmitted, by a transmitter using packetizing, to an external hub.

In a further embodiment, the processing circuitry further includes a control circuit configured to transmit the digital signal via the wireless transmitter in accordance with a control code.

In a further embodiment the analog-to-digital converter uses a successive approximation register (SAR ADC) architecture.

In a still further embodiment, the first RF signal is modulated using pulse width modulation-amplitude shift keying (PWM-ASK).

In another further embodiment, the processing circuitry further includes an amplifier configured to amplify the electric signal, wherein the amplifier is communicatively coupled to the at least one sensing electrode and the SAR ADC.

In another embodiment, the processing circuitry further includes: a first low-dropout regulator (LDO) coupled to the power harvesting circuit, wherein the first LDO is configured to supply energy to the processing circuitry; and a second low-dropout regulator coupled to the power harvesting circuit configured to supply energy to the wireless transmitter.

In another embodiment, the first RF signal is modulated with the control code.

In another embodiment, the first RF signal frequency is between 1 and 100 Mhz.

In still another embodiment, the second RF signal frequency is between 100 and 10000 Mhz.

In another embodiment, the power harvesting circuit includes a five-stage passive rectifier.

In yet another embodiment, the second RF frequency is transmitted to an external wearable device.

In another embodiment, the second RF frequency is processed and filtered on an external wearable device to reduce noise.

In another embodiment, the second RF frequency is converted to base-band and frequency contents below 1 Hz and higher than 10 KHz are removed.

In still another embodiment, an adjustable parameter of the device is selected from the group consisting of its frequency of operation, power consumption, number of bits, and duty-cycle.

In yet another embodiment, the output from the ADC is serialized through parallel-to-serial (P2S) logic.

In another embodiment, the sensing device includes a direct power oscillator and an LC oscillator.

In still yet another embodiment, the sensing device includes: a power management unit (PMU) configured to set an operating mode and maintain a minimum voltage; and a receiver circuitry block configured to provide energy from the first RF signal to the power harvesting circuit.

In a further embodiment, the PMU is configured to control the wireless transmitter to operate on a duty cycle based upon a current amount of energy stored in a storage capacitor.

In another further embodiment, the wireless transmitter includes a data modulator circuit, the data modulator circuit configured to generate the second RF signal using DC voltage received from the power management unit.

In a further embodiment, the sensor device includes an N-well N-type metal-oxide-semiconductor (N-well NMOS) transistor, wherein the N-well NMOS transistor is configured to regulate the DC voltage.

In another embodiment, the wireless receiver includes a loop antenna and a capacitor.

In another embodiment, the output from the ADC is converted to return-to-zero (RZ) format to generate a pulse symbol.

In yet another embodiment, the clock extraction includes: demodulating, the first RF signal to obtain an envelope signal, wherein the demodulation is performed using at least one of an envelope detector and a self-mixing principle; filtering, using a low pass filter, the envelope signal; recovering, using a comparator, one or more crossing points between the filtered envelope signal and a reference signal; and generating, using the comparator, a clock signal from the crossing points.

In a further embodiment, the clock extraction further includes removing noise from the clock signal with a Schmitt trigger.

In another further embodiment, operating modes are selected based on the first RF signal.

In another embodiment, the clock signal is used to set rates for at least one of acquiring signal samples and wirelessly receiving data transmissions.

In another embodiment, the clock signal is used to synchronize a reset signal for the sensing device to a supply domain for one or more sensors on the sensing device.

In yet another embodiment, the transmitter using packetizing is configured to transmit data with an 8-bit preamble indicating a starting point of the data.

One embodiment includes a biopotential sensing device. The biopotential sensing device includes: a wireless receiver configured to receive a first radio frequency (RF) signal; one or more wireless transmitters configured to transmit a second RF signal, where the first RF signal and the second RF signal have different frequencies; and a processing circuitry. The processing circuitry includes a power harvesting circuit configured to harvest energy from the first RF signal. The processing circuitry includes a clock recovery circuit configured to extract a clock signal from the first RF signal. The processing circuitry includes at least one sensing electrode configured to record a cardiac signal describing cardiac activity. The processing circuitry includes a successive approximation register analog-to-digital converter (SAR ADC) communicatively coupled to the clock recovery circuit configured to convert the cardiac signal into a digital signal. The processing circuitry includes a control circuit configured to transmit the digital signal via the wireless transmitter in accordance with a control code. The processing circuitry includes a first low-dropout regulator coupled to the power harvesting circuit configured to supply energy to the processing circuitry. The processing circuitry includes a second low-dropout regulator coupled to the power harvesting circuit configured to supply energy to the wireless transmitter.

In a further embodiment, the output from the ADC is converted to return-to-zero (RZ) format to generate a pulse symbol.

In another further embodiment, the clock signal is used to synchronize at least one wireless transmitter with the conversion of the cardiac signal into the digital signal, and output from the ADC is serialized and transmitted, by a transmitter using packetizing, to an external hub.

In a further embodiment, the output from the ADC is serialized through parallel-to-serial (P2S) logic.

In another embodiment, the first RF signal is modulated with at least one of the control code and a use of pulse width modulation-amplitude shift keying (PWM-ASK).

In another embodiment, the first RF signal frequency is between 1 and 100 Mhz.

In yet another embodiment, the second RF signal frequency is between 100 and 10000 Mhz; the second RF signal frequency is converted to base-band; and frequency contents between 1 Hz and higher than 10 KHz are removed.

In another embodiment, the second RF signal frequency is transmitted to, processed on, and filtered on an external wearable device.

In still another embodiment, the power harvesting circuit includes a five-stage passive rectifier; and an amplifier configured to amplify the cardiac signal, and the amplifier is communicatively coupled to the at least one sensing electrode and the SAR ADC.

In another embodiment, the biopotential sensing device includes a direct power oscillator and an LC oscillator.

In yet another embodiment, the biopotential sensing device includes: a power management unit (PMU) configured to set an operating mode and maintain a minimum voltage; and a receiver circuitry block configured to provide energy from the first RF signal to the power harvesting circuit.

In a further embodiment, the PMU is configured to control the wireless transmitter to operate on a duty cycle based upon a current amount of energy stored in a storage capacitor.

In another further embodiment, the wireless transmitter includes a data modulator circuit, the data modulator circuit configured to generate the second RF signal using DC voltage received from the power management unit.

In a further embodiment, the biopotential sensing device includes an N-well N-type metal-oxide-semiconductor (N-well NMOS) transistor, wherein the N-well NMOS transistor is configured to regulate the DC voltage.

In another embodiment, the wireless receiver includes a loop antenna and a capacitor.

In another embodiment, operating modes are selected based on the first RF signal.

In another embodiment, the clock extraction includes: demodulating, the first RF signal to obtain an envelope signal, wherein the demodulation is performed using at least one of an envelope detector and a self-mixing principle; filtering, using a low pass filter, the envelope signal; recovering, using a comparator, one or more crossing points between the filtered envelope signal and a reference signal; and generating, using the comparator, a clock signal from the crossing points.

In a further embodiment, the clock extraction further includes removing noise from the clock signal with a Schmitt trigger.

In another embodiment, the clock signal is used to set rates for at least one of acquiring signal samples and wirelessly receiving data transmissions.

In another embodiment, the clock signal is used to synchronize a reset signal for the biopotential sensing device to a supply domain for one or more sensors on the biopotential sensing device.

In yet another embodiment, the transmitter using packetizing is configured to transmit data with an 8-bit preamble indicating a starting point of the data.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIGS. 1A-1B illustrate an integrated circuit (IC) architecture and corresponding timing diagram in accordance with an embodiment of the invention.

FIG. 2 illustrates a circuit schematic of an energy harvester and a voltage regulator unit that converts AC voltage to regulated DC voltage in accordance with an embodiment of the invention.

FIG. 3 illustrates a circuit schematic of a low dropout (LDO) regulator with a capacitor-less (cap-less) design dedicated to supplying power to the transmitter (TX) block (LDO_TX) in accordance with an embodiment of the invention.

FIGS. 4A-4B illustrate a circuit schematic of a clock and baseband envelope detector circuit in accordance with an embodiment of the invention.

FIG. 5A illustrates a circuit schematic of differential capacitive coupled instrumentation amplifier (IA) in accordance with an embodiment of the invention.

FIG. 5B illustrates a circuit schematic of a low-power successive approximation register (SAR) analog-to-digital converter (ADC) in accordance with an embodiment of the invention.

FIGS. 6A-6B illustrate a circuit implementation of the TX block and data interface in accordance with an embodiment of the invention.

FIGS. 7A-7B illustrates a circuit schematic of a local oscillator-less RX implementation and timing diagram in accordance with an embodiment of the invention.

FIGS. 8A-8B illustrate packet structure diagrams for uplinks and downlinks in accordance with an embodiment of the invention.

### DETAILED DESCRIPTION

Turning now to the drawings, a wirelessly powered, battery-less and closed-loop biopotential recording system for use in implantable leadless cardiac monitoring applications in accordance with many embodiments of the invention is illustrated. The requirement of batteries to function tends to make implantable cardiac devices bulky. For example, pacemakers must be implanted under a patient's collarbone and further away from the heart due to lack of space, and the leads which deliver stimulation to the heart can cause issues as well. Potential dangers include catastrophic battery failures and clogged arteries, which are undesirable. Sensing-specific wearable devices may be smaller, but can still suffer from large power solutions and other flaws.

Energy harvesting technologies may be used to address battery-related issues. Such technologies may include, but are not limited to piezoelectric harvesting and radio frequency (RF) energy harvesting, e.g. via RF induction. However, among possible issues are the fact that the piezoelectric method relies on the ability of certain materials to generate an electric charge in response to applied mechanical stress. A large element area is required to generate a stable charge, which may not be feasible inside the chest of a patient. Multisite pacing may often be an issue as there is not enough area to implant multiple pacemakers for patients who are less responsive, or not responsive, to cardiac synchronization therapy. Recording and parameter adjustments are often inadequate due to the lack of energy.

RF energy harvesting (RFEH) may capture and convert electromagnetic energy into usable DC voltages. Though low-frequency waves may have lower attenuation on the human body, the necessary antenna size can increase with the usage of low-frequency signals. Further, conventional RFEH systems may lack advanced control since the integrated circuit (IC) is not integrated with sophisticated on-chip logic. Systems and methods described herein address the limitations of the energy harvesting methodologies above, and are able to achieve fully battery-less operation through wireless energy harvesting to be used in implantable leadless cardiac applications.

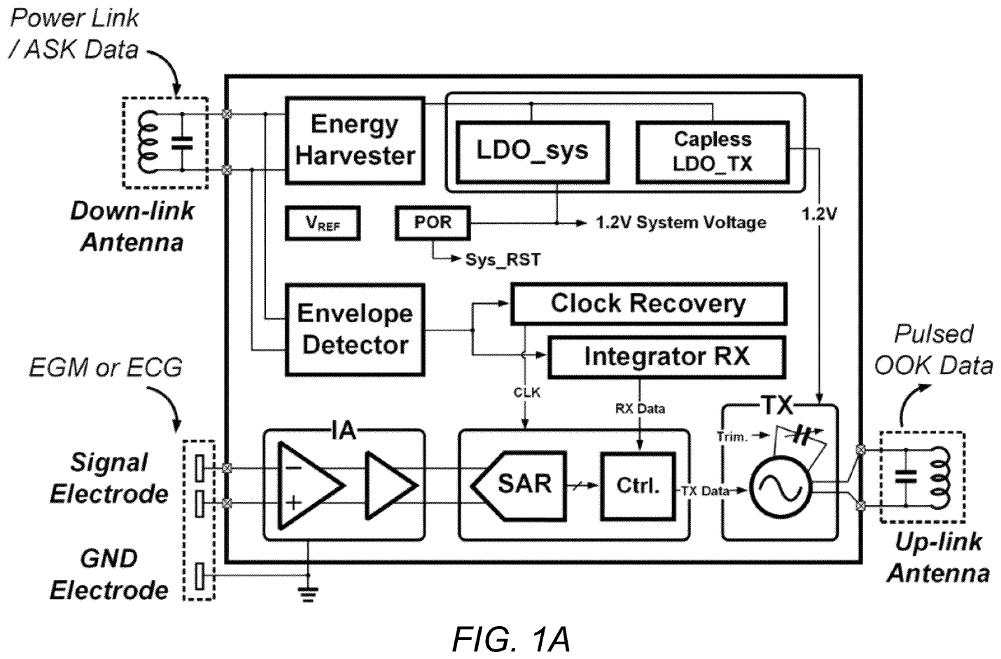

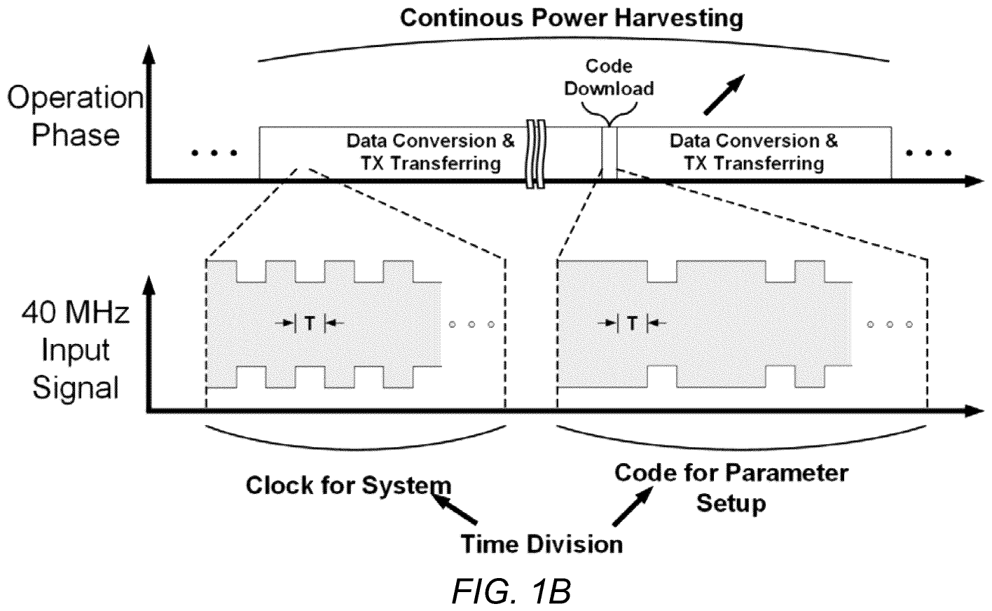

FIGS. 1A-1B illustrate an integrated circuit (IC) architecture and corresponding timing diagram in accordance with some embodiments of the invention. IC architectures in such embodiments may include but are not limited to a radio frequency (RF) energy harvester, power management unit, biopotential-acquisition instrument amplifier (IA), analog-to-digital converter (ADC), clock recovery unit, receiver, and transmitter (TX). ICs may be controlled by one or more externally provided clocks, in order to eliminate dependency on local oscillators. One or more energy harvesters and power management units may ensure the stable provision of energy sources to the system. A cost-efficient clock recovery unit and data demodulator receiver (RX) for the downlink may enable functionality under challenging batteryless environments without relying on local oscillators. Systems may further include biopotential acquisition chains including but not limited to signal channel instrument amplifiers (IAs) for cardiac monitoring. Additionally, low-power TXs may transfer acquired data through uplink channels to external wireless readers.

An IC system architecture implementation, in accordance with a number of embodiments of the invention, is depicted in FIG. 1A. In many embodiments, ICs may be constructed with components including but not limited to: 1) A power harvesting system that receives RF energy through a wireless link. 2) A low dropout (LDO) voltage regulator split into two separate LDOs with: LDO_sys designed with conventional LDO architecture dedicated to supplying power to the system; and an LDO_TX designed with cap-less architecture dedicated to supplying power to TX Antenna to facilitate output transmission. 1) A power harvesting system that receives RF energy through a wireless link. 2) A low dropout (LDO) voltage regulator split into two separate LDOs with: LDO_sys designed with conventional LDO architecture dedicated to supplying power to the system; and an LDO_TX designed with cap-less architecture dedicated to supplying power to TX Antenna to facilitate output transmission. 3) A clock and data baseband envelop detector circuit that provides an accurate clock signal for the system. 4) A single-channel, fully differential capacitive-coupled instrumentation amplifier (IA) that increases the voltage amplitude. 5) A low-power successive approximation register (SAR) analog-to-digital converter (ADC) that converts the amplified signal by the IA to a digital signal. 6) a wireless transmitter that has its operation synchronized with the SAR ADC.

Systems operating in accordance with numerous embodiments may utilize additional components including but not limited to sensor electrodes. Sensor electrodes may be configured to record electric signals as at least one of voltage, current, and electric charges. For integrated digital logic, signals to initial resets may be utilized. Specifically, reset signals may be synchronized with the supply domain for the miniaturized sensor electrode configuration. ICs may further incorporate two input ports connected with a downlink antenna and a signal electrode. Downlink antennae may be used to receive wireless data and power. Signal electrodes may be applied to cardiac signal sensing. Architectures may further include analog signal chain grounds (GND) connected to GND electrodes. In accordance with numerous embodiments of the invention, frequency planning of the ICs can be compatible with industrial, scientific, and medical (ISM) radio bands. ICs may incorporate two separated channels. For example, an IC may use center frequency 40.68 MHz for downlink and 915 MHz for uplink.

As mentioned above, architectures may incorporate energy harvesters that power various regulators. For example, as in FIG. 1A, energy harvesters may provide DC power to separated low-dropout regulators (LDO) that may be used for the transmitter (TX) and the rest of the circuit. Energy harvester peak conversion efficiency may show more than 40% peak efficiency. The total power consumption of ICs operating in accordance with some embodiments of the invention may go as low as 8.8 μW for configurations operating with 100 kHz clock frequency. Signal acquisition paths operating in ICs may integrate, but are not limited to single-channel, differential capacitive-coupled IAs and SAR-ADCs. Parallel data output from ADCs operating in accordance with numerous embodiments may be serialized and transmitted to external hubs through TXs utilizing packetizing, as will be discussed further below. Transmitter output may drive the uplink antenna coil. In doing so, pulsed on-off keying (OOK) data may be streamed to external hubs.

FIG. 1B illustrates different modes of operations for systems operating in accordance with several embodiments of the invention. Systems can be divided into conversion transferring and code-download modes. Modes may be selected based on envelope modulated data obtained from data baseband envelop detector circuits. In code-download modes, the RX may receive and upload the control code to the system(s). In conversion transfer modes, baseband signals may provide accurate clock signals for system operation (“system clocks”). System clocks may become references for timing generation and enable synchronized operations between systems and TX.

In accordance with certain embodiments of the invention, clock signals may be received externally. Architectures in accordance with a number of embodiments of the invention may support duty-cycled and/or continuous operation. Since the clock signal may set the signal acquisition sampling rates and/or wireless data rates, systems may depend of the timing source being accurate and non-power intensive. Moving the timing facilitation to external sources may enable systems operating in accordance with numerous embodiments to improve the system specifications and energy efficiency. Moreover, the burden of code download may be minimized for long-term monitoring applications, as evidenced by the short time slot of FIG. 1B.

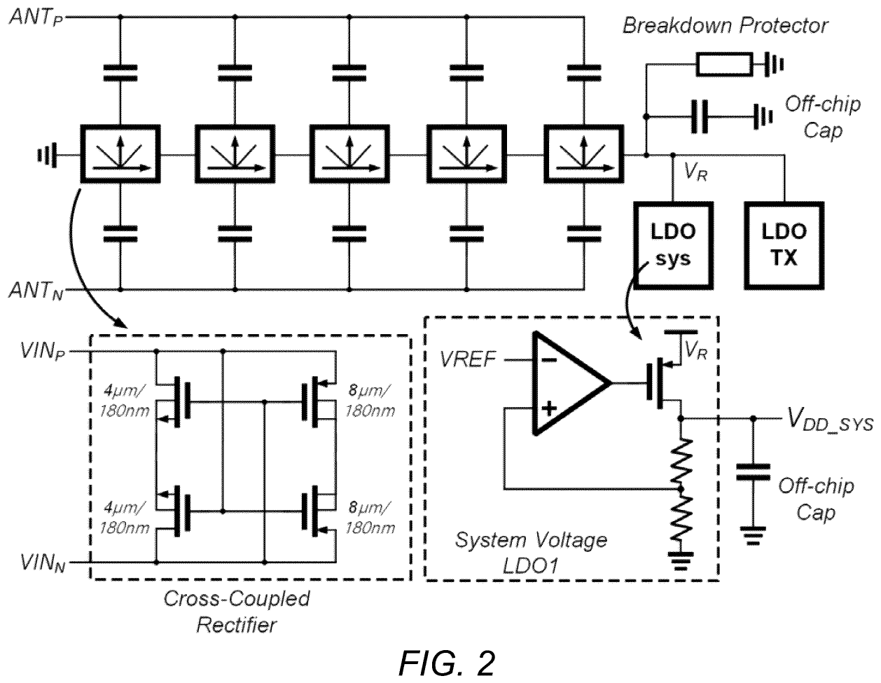

Power sources of systems operating in accordance with numerous embodiments of the invention may be replaced by energy harvesters and voltage regulators, as illustrated in FIG. 2. Energy harvesters and voltage regulator units may be used to convert AC voltage to regulated DC voltage. In a number of embodiments, energy harvesters may be fabricated with integrated five-stage passive rectifiers. Five-stage passive rectifiers may include but are not limited to cross-coupled complementary metal-oxide-semiconductor (CMOS) rectifiers. Passing through each rectifier stage, stacked DC voltages may generate voltage gaps between transistor bodies and sources. To reduce variation in the threshold voltages and leakage, a deep N-well N-type metal-oxide-semiconductor (NMOS) transistor may be incorporated. Additionally or alternatively, harvester output electrodes may integrate diode stacks as protection blocks to prevent transistor breakdown in the event of high voltage surges. The rectifiers may allow systems to obtain regulated DC voltage, deep N-Well NMOS transistors may mitigate efficiency degradation, and/or protection blocks may prevent transistor breakdown under voltage surges.

The output of energy harvesters may be fed to the inputs of two LDOs: system LDOs (LDO_sys) and transmitter LDOs (LDO_TX). In dynamic power profiles of systems operating in accordance with numerous embodiments, the transmitter may be the block consuming the most current. Therefore, for certain systems, the system and TX power domains can be isolated with separated LDOs. One possible effect may be to prevent the noise from the power supply affecting TX outputs and minimize the effect of potential coupling through the power domain. LDO_sys (System LDOs) may be fabricated from conventional LDO architectures with one off-chip capacitor. The voltage reference for the LDO_sys can take the form of a constant-gₘ bias circuit, which may be shared throughout the entire system to reduce further power consumption in accordance with many embodiments of the invention.

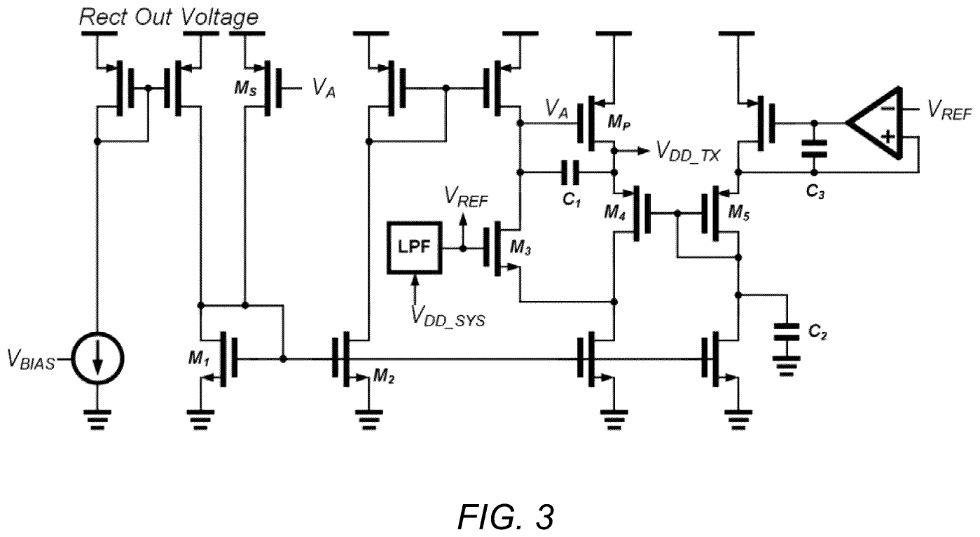

FIG. 3 illustrates a circuit schematic of an LDO_TX in accordance with certain embodiments of the invention. In accordance with a variety of embodiments, the LDO_TX may adopt a cap-less architecture to reduce the number of external components. Cap-less architectures may therefore ensure a small overall system form factor. As mentioned above, voltage references may be shared throughout entire systems, which may mean that the voltage reference for LDO_TX can be sampled from the filtered output of LDO_sys. In accordance with numerous embodiments, the outputs of LDO_sys and LDO_TX may therefore be maintained at the same level. A sampling MOSFET Mₛ can be included to enable adaptive biasing, which can allow biasing currents of M₁ and M₂ to be adjusted during load transitions.

The TX for systems operating in accordance with some embodiments may be a high-duty cycled block. In such cases, there may be two adaptive requirements: quiescent current saving in the off status and fast transient response in the active state. In the design of the corresponding LDOs, there may be more rapid responses related to loop bandwidth, which can require a higher quiescent current. In accordance with such designs, adaptive loop bandwidth control techniques may be applied.

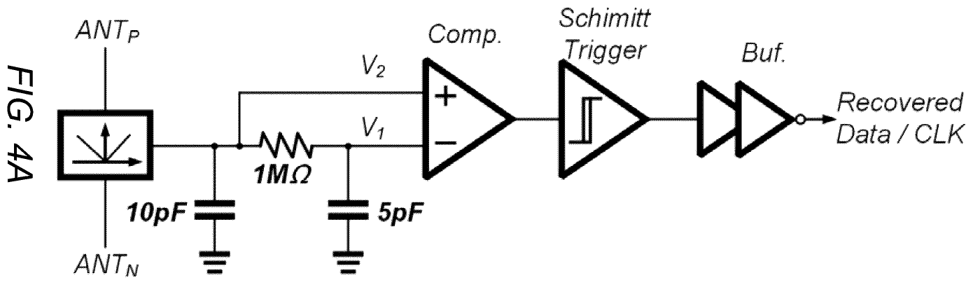

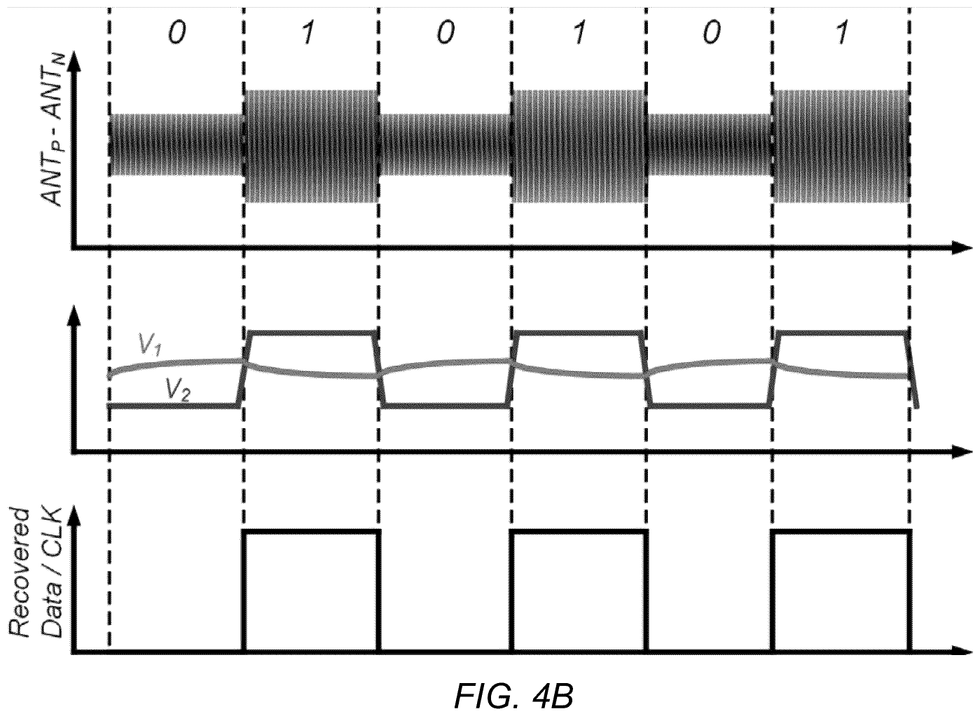

FIGS. 4A-4B illustrate a circuit schematic of the clock and data baseband envelop detector circuit in accordance with many embodiments of the invention. As indicated above, systems operating in accordance with some embodiments of the invention may have two modes: a code-download mode and a conversion-transferring mode. When download mode is active, the system can detect the signal of input low-frequency signals through the antenna (ANT_{P}-ANT_{N}) that determines the next operation of the system. Signals may be demodulated through the self-mixing principle. Additionally or alternatively, input code may be based on pulse width modulation-amplitude shift keying (PWM-ASK) to save power for other components. Keying methods may vary for different embodiments of this invention. In the conversion-transferring mode, the system may demodulate the baseband data with envelope detectors to provide clocks for the overall system. The input signals coming through the antennae may include, but is not limited to, a power carrier and a low-frequency amplitude modulated signal. The low-frequency amplitude modulated signal may, for instance, have a signal frequency smaller than 100 KHz.

The aforementioned envelope, as well as a reference signal, may be generated through single-stage rectifiers configured in accordance with a variety of embodiments. Systems in accordance with numerous embodiments, while using the passive low pass filter, may obtain crossing points between slowly varying (V1) reference signals and an envelope (V2) signals. Such crossing points may be recovered and used as clock signals. Recovery may occur through the comparator. Additionally or alternatively, envelope signals and reference signals may be converted to digital signals through comparator-Schmitt trigger chains to recover the clock.

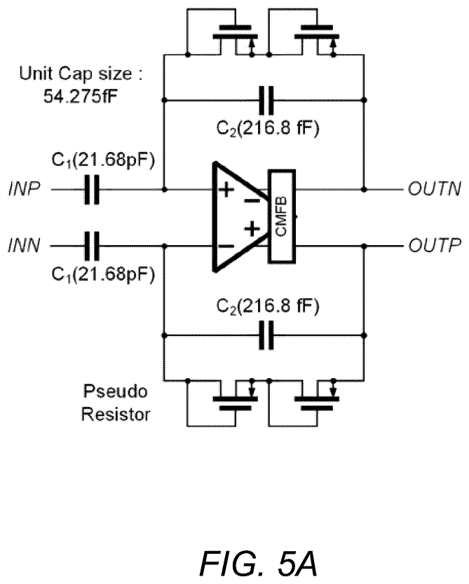

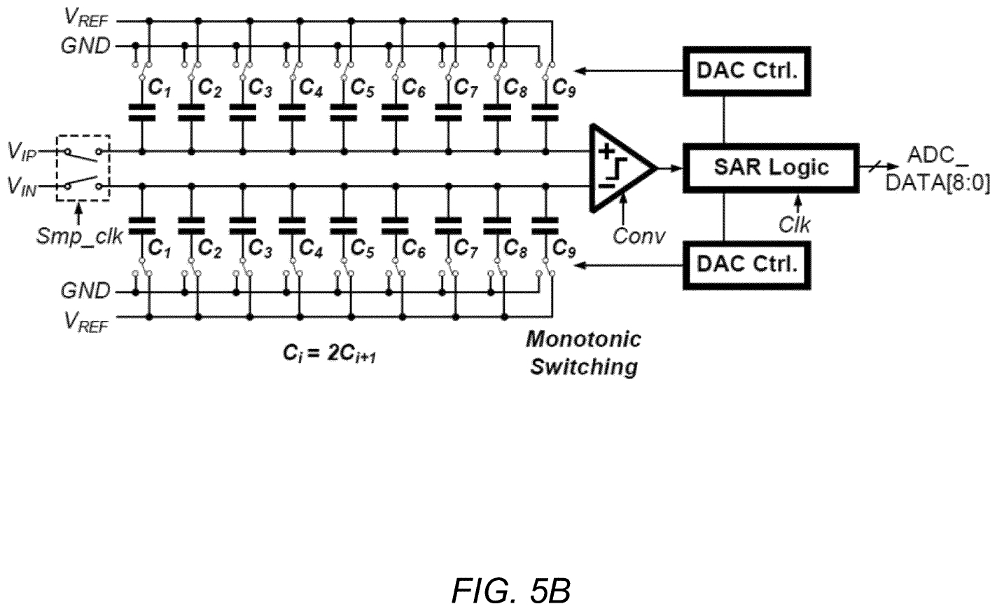

A circuit schematic of the differential capacitive-coupled IA in accordance with some embodiments of the invention is illustrated in FIG. 5A. The AC gain of the IA in FIG. 5A, may be determined by the sizing ratio of C₁ (21.68 pF) and C₂ (216.8 fF). The capacitors may be designed to provide specific amplification gains (e.g., 40-dB). IAs may have multiple unit cap sizes for matching purposes. In a number of embodiments, amplifier biasing may be accomplished through common-mode feedback (CMFB) on pseudo resistor feedbacks that set the high cut-off frequency. The operational transconductance amplifier (OTA) may be designed with two-stage amplifiers using resistive feedback common-mode sensing paths. The output of such IAs may be fed to drive the SAR ADC. SAR ADCs may be used to amplify the biopotential signal and digitize it so the acquired data can be transmitted robustly.

FIG. 5B illustrates a circuit schematic of low-power SAR ADCs configured in accordance with several embodiments of the invention. Emphasis may be placed on keeping ADCs simplistic with regard to hardware design. Additionally or alternatively, systems operating in accordance with several embodiments of the invention may emphasize keeping ADCs energy efficient by adopting monotonic capacitor-switching techniques. ADC operation can be synchronized to system clocks, which can be generated from clock recovery units. Data converted by ADCs operating in accordance with certain embodiments of the invention may be passed to TX blocks.

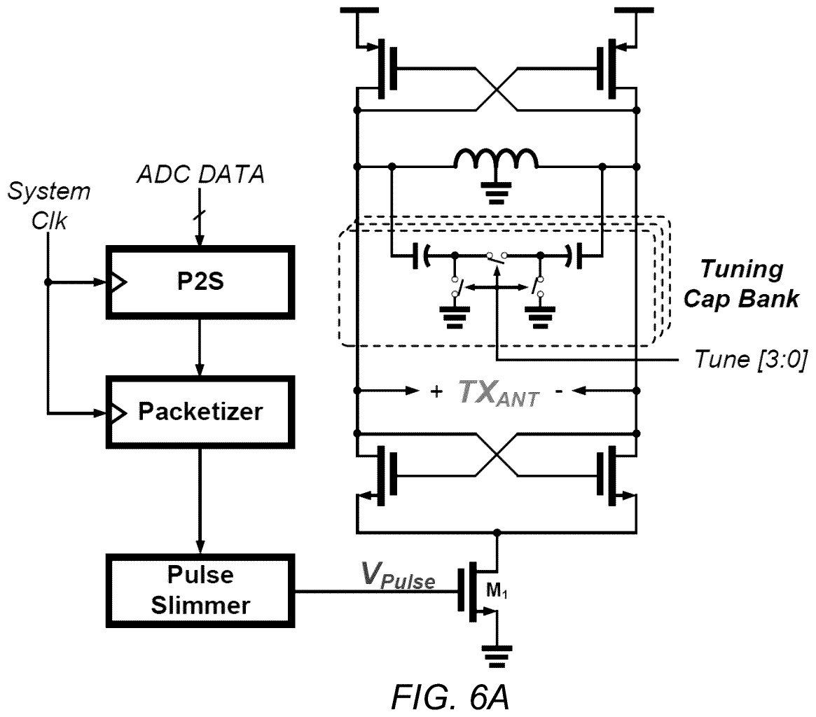

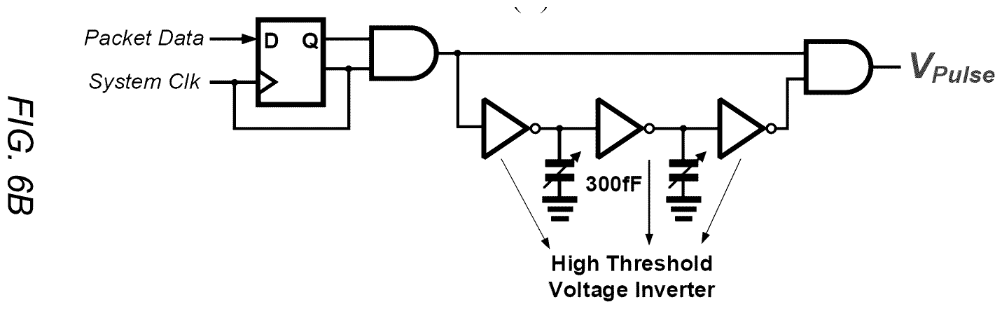

FIG. 6A illustrates the schematic of wireless TX block implementation, alongside a data interface, in accordance with many embodiments of the invention. Asymmetrical power budgets between the implantable transmitters and external hubs, may benefit from the use of direct power oscillators. LC oscillator-based power amplifiers (PAS) may be adopted to reduce energy consumption and simplify hardware.

FIG. 6B illustrates the packetizer output stage and pulse slimmer circuit diagram in accordance with numerous embodiments of the invention. Wireless transmitters configured in accordance with numerous embodiments may operate synchronously with the SAR ADC. In such cases, the converted data from the ADC may be serialized through parallel-to-serial (P2S) logic and/or modulated as packet structures through packetizing blocks. Packetizing blocks may use On-Off-Keying (OOK) and/or ultra-wideband designs for further reducing power consumption.

In many cases, data may be converted to return-to-zero (RZ) format to generate pulse symbols. Delay flip-flops and AND gates may be used to derive values for synchronized packet data. In accordance with many embodiments, the oscillator output frequency can be varied by methods including but not limited to, process variation and passive antenna connection. The output driving frequency of POs can be controlled by on-chip capacitor banks inserted between the output electrodes.

Post-fabrication frequency tuning may be accomplished through binary-weighted tuning capacitor banks between the output ports. A control Tune [3:0] code can be set wirelessly by decoding the received data. In many embodiments, TX antennae may utilize, but are not limited to, resonant loops (which may have a frequency of 915 MHz) in order to improve transmission efficiency and reduce form factors of the overall system. Capacitor banks may be composed of, but are not limited to, offset capacitors (e.g., 3 pF capacitance) and/or binary-weighted caps (e.g., resolution steps of 206.3 fF). These components may be used to tune free-running frequency to the above resonant loop frequency using the code downloaded.

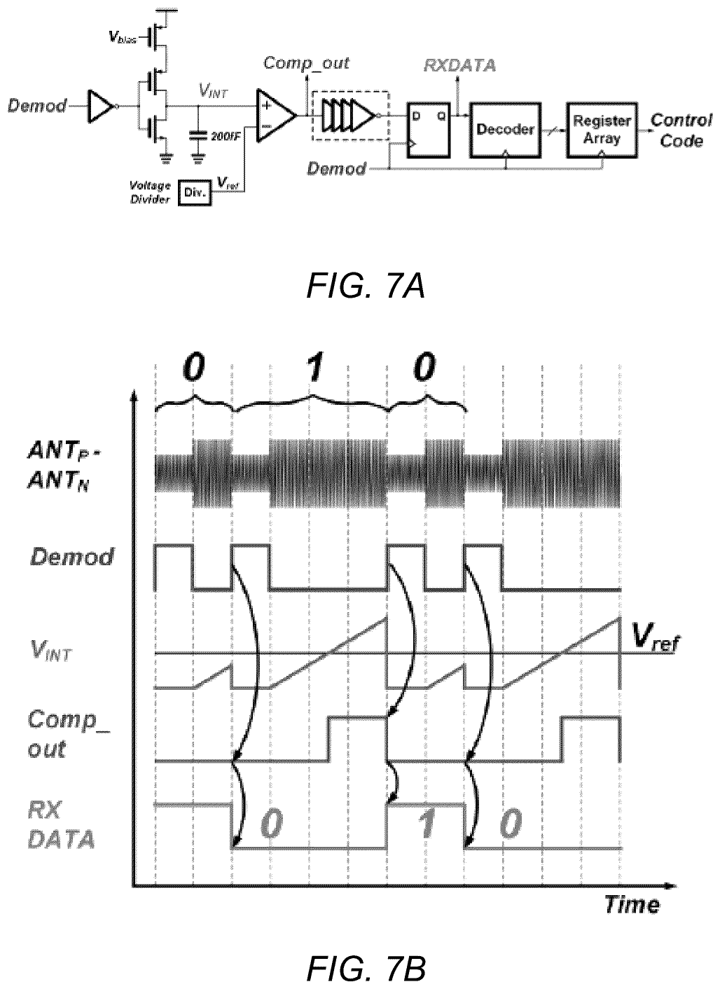

FIGS. 7A-7B illustrate a circuit schematic of a local oscillator-less RX implementation and timing diagram in accordance with various embodiments of the invention. The integrated receivers may enable wireless controllability in systems operating in accordance with various embodiments. Digital transition signals shared from clock recovery units may be input into RXs. Demod signals may be used to control the accumulation and evaluation of VINT voltage electrodes incorporated into the RX. Baseband data may be modulated through pulse-width modulated amplitude shift keying (PWM-ASK). The two bits, 0 and 1, can be distinguished through higher amplitude lasting times. Low to high transitions may provide timing transition edges for each operation.

The transition edges of recovered clock signals may be used for sampling clocks. Such sampling clocks may also be fed to SAR ADCs to reduce, thereby adding additional conversion clocks. Sampling clocks, from the transition edges in the recovered clock signal above, may be fed such that they can proceed immediately after sampling the analog data. The voltage from sampling electrodes may converge from half voltage references to GND based on switching algorithms. In some embodiments, PMOS strong-arm latches may be adopted at the input to minimize the offset. Binary-weighted caps may be designed as metal-insulator-metal (MIM) arrays. The unit capacitor size may be 35.6 fF, while the layout may be implemented as common centroids.

The time duration of symbol differences may indicate charge integration time differences. Vbias may be used to limit the current flow to charge 200 fF MIM capacitor(s) in the VINT electrode(s). Additionally or alternatively, in the other branch, the voltage divider, composed of multiple diode-connected PMOS chains, may provide half the VDD voltage. In accordance with several embodiments, continuous comparators may detect the crossing point of two signals and generate Comp_out signals. The Comp_out signals may be used to indicate recovered transferred baseband data. Transition edges of Demod may be used for sampling clocks configured in accordance with some embodiments. The Comp_out data can go through a low-power delay line that may push the transition to prevent metastability induced from potential transition overlap. The recovered payload data and the sampled signal, demodulated from the packet at the decoder, can be updated to the register array.

From system perspectives, one of the critical elements of controllability may be accurate clock sampling timing. For accurate data demodulation and sampling, clock-data-recovery (CDR) and/or coherent Costas loops may be required. Approaches in accordance with numerous embodiments may be beneficial in terms of power consumption, since they may not require closed feedback loop configurations. Systems may have relatively narrow operating symbol rates, which can come from the voltage accumulation slope in VINT electrodes. However, in many embodiments, the programming time may be negligible compared to the data streaming mode in nominal operation. Moreover, the burden on the external hub transmitter may be insignificant, since the data rate can be easily adjusted. Based on past measurements, operations in accordance with some embodiments can be performed with 1 kHz to 10 kHz symbol rates. The prototype design illustrated in FIG. 7A may use the received data for oscillation frequency adjustments in the uplink TX of FIG. 5A. Further, the integrated RX can be utilized with higher complexity by adding expanded controllability.

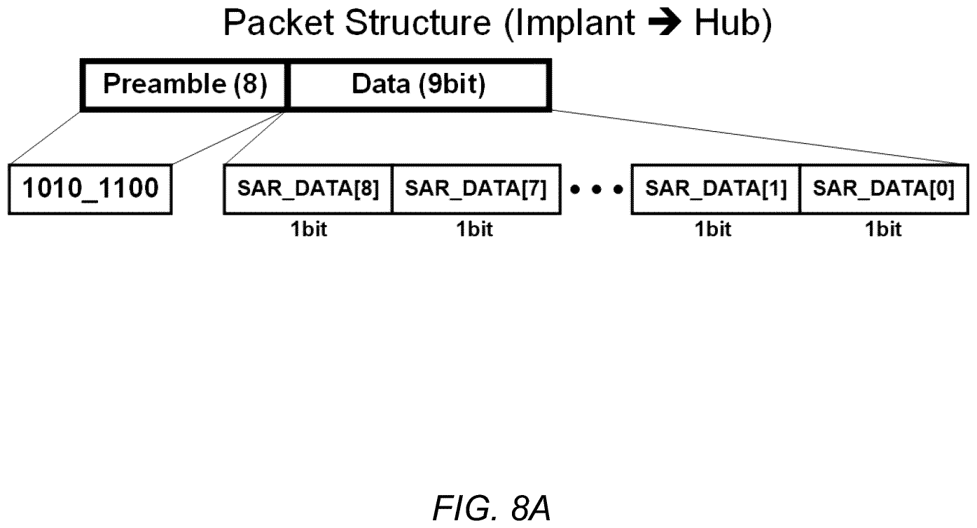

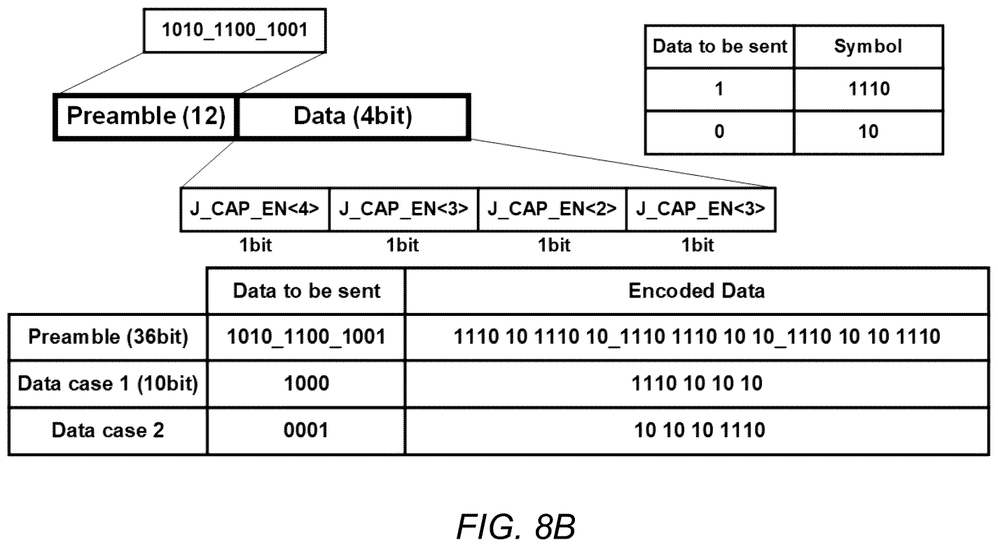

FIGS. 8A-8B illustrate packet structure diagrams in accordance with many embodiments of the invention. Packet structures may be used in data communication in uplink (Implant to Hub) and downlink (Hub to Implant) situations. In uplink and/or downlink scenarios, packets may be used to extract transferred data to distinguish required information under signal streams. Higher-order packet modulation may allow reliable data demodulation, decrease effective data rates, and decrease the possibility of data loss. In accordance with certain embodiments, packet structure design can promote energy efficiency and data reliability.

FIG. 8A depicts an uplink packet structure. Packet structures in accordance with a number of embodiments may allocate single sample data per packet to ensure better data recovery capability. The 8-bit preambles of packets may indicate the starting point of data. Additionally or alternatively, ADC data may be transmitted to the external hubs directly and without compression encoding.

FIG. 8B depicts a downlink data packet structure and encoding pattern. In the uplink, as many as 12 bits may be adopted. Additionally or alternatively, four trimming bits may be included as data payloads. The trimming bits can allow fully wireless control of the TX oscillation frequency.

Although specific methods of fabricating a wirelessly powered, battery-less closed loop biopotential recording ICs for implantable leadless cardiac monitoring applications are discussed above, many different fabrication methods can be implemented in accordance with many different embodiments of the invention. Further, as can readily be appreciated, although the above is written with a focus on the heart but systems and methods described herein can be used in any implantable medical applications and as well as any wirelessly powered devices that are not implantable. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

1. A sensing device, comprising:
a wireless receiver configured to receive a first radio frequency (RF) signal;
one or more wireless transmitters,
wherein at least one wireless transmitter is configured to transmit a second RF signal, and
wherein the first RF signal and the second RF signal have different frequencies; and
a processing circuitry, comprising:
a power harvesting circuit configured to harvest energy from the first RF signal;
a clock recovery circuit configured to extract a clock signal from the first RF signal;
at least one sensing electrode configured to record an electric signal as at least one of a voltage, current, and electric charge; and
an analog-to-digital converter (ADC) communicatively coupled to the clock recovery circuit,
wherein the ADC is configured to convert the electric signal into a digital signal,
wherein the clock signal is used to synchronize at least one wireless transmitter with the conversion of the electric signal into the digital signal, and
wherein output from the ADC is serialized and transmitted, by a transmitter using packetizing, to an external hub.

2. The sensing device of claim 1, wherein the processing circuitry further comprises at least one of:
a control circuit configured to transmit the digital signal via the at least one wireless transmitter in accordance with a control code;
a first low-dropout regulator (LDO) coupled to the power harvesting circuit, wherein the first LDO is configured to supply energy to the processing circuitry; or
a second low-dropout regulator coupled to the power harvesting circuit configured to supply energy to the at least one wireless transmitter.

3. The sensing device of claim 2, wherein the analog-to-digital converter uses a successive approximation register (SAR ADC) architecture.

4. The sensing device of claim 3, the processing circuitry further comprises an amplifier configured to amplify the electric signal, wherein the amplifier is communicatively coupled to the at least one sensing electrode and the SAR ADC.

5. The sensing device of claim 2, wherein the first RF signal is modulated using at least one of:
the control code; or
pulse width modulation-amplitude shift keying (PWM-ASK).

6. (canceled)

7. The sensing device of claim 1, wherein:
a signal frequency of the first RF signal is between 1 and 100 Mhz; and
a signal frequency of the second RF signal is between 100 and 10000 Mhz.

8. (canceled)

9. The sensing device of claim 1, wherein the power harvesting circuit comprises a five-stage passive rectifier.

10. (canceled)

11. The sensing device of claim 1, wherein the second RF signal undergoes at least one of being:
transmitted, to an external wearable device;
processed and filtered on the external wearable device to reduce noise; or
converted to base-band, wherein frequency contents below 1 Hz and higher than 10 KHz are removed.

12. (canceled)

13. (canceled)

14. The sensing device of claim 1, wherein an adjustable parameter of the sensing device is selected from the group consisting of its frequency of operation, power consumption, number of bits, and duty-cycle.

15. The sensing device of claim 1, wherein the output from the ADC undergoes at least one of being:
converted to return-to-zero (RZ) format to generate a pulse symbol; or
serialized through parallel-to-serial (P2S) logic.

16. The sensing device of claim 1, further comprising at least one of:
a direct power oscillator and an LC oscillator; or
a loop antenna and a capacitor for the wireless receiver.

17. The sensing device of claim 1, further comprising:
a power management unit (PMU) configured to set an operating mode and maintain a minimum voltage; and
a receiver circuitry block configured to provide energy from the first RF signal to the power harvesting circuit.

18. The sensing device of claim 17, wherein the at least one wireless transmitter comprises a data modulator circuit, the data modulator circuit configured to generate the second RF signal using DC voltage received from the PMU.

19. The sensing device of claim 18, further comprising an N-well N-type metal-oxide-semiconductor (N-well NMOS) transistor, wherein the N-well NMOS transistor is configured to regulate the DC voltage.

20. (canceled)

21. The sensing device of claim 17, wherein the PMU is configured to control the at least one wireless transmitter to operate on a duty cycle based upon a current amount of energy stored in a storage capacitor.

22. (canceled)

23. The sensing device of claim 1, wherein the clock extraction comprises:
demodulating the first RF signal to obtain an envelope signal, wherein the demodulation is performed using at least one of an envelope detector and a self-mixing principle;
filtering, using a low pass filter, the envelope signal;
recovering, using a comparator, one or more crossing points between the filtered envelope signal and a reference signal; and
generating, using the comparator, the clock signal from the one or more crossing points.

24. The sensing device of claim 23, wherein the clock extraction further comprises removing noise from the clock signal with a Schmitt trigger.

25. The sensing device of claim 1, wherein the clock signal is used for at least one of:
setting rates for at least one of acquiring signal samples and wirelessly receiving data transmissions; or
synchronizing a reset signal for the sensing device to a supply domain for one or more sensors on the sensing device.

26. (canceled)

27. The sensing device of claim 1, wherein the transmitter using packetizing is configured to transmit data with an 8-bit preamble indicating a starting point of the data.

28. The sensing device of claim 1, wherein operating modes are selected based on the first RF signal.

29-49. (canceled)
